SEQUENCE LISTING (24 sequences)

SEQ ID NO: 1 - DNA, 66 bp, Artificial Sequence
  random oligonucleotide
tgagatctggatccgttcggggaagggaaggtgaaagcaagaattaccgtcctacgaattcagctg

SEQ ID NO: 2 - DNA, 66 bp, Artificial Sequence
  random oligonucleotide
tgagatctggatccgttcgacaaaacggtaaaaaagcggtagattaccgtcctacgaattcagctg

SEQ ID NO: 3 - DNA, 66 bp, Artificial Sequence
  random oligonucleotide
tgagatctggatccgttcgacaaaacggtaaaactaaaggtaactgacgtcctacgaattcagctg

SEQ ID NO: 4 - DNA, 64 bp, Artificial Sequence
  random oligonucleotide
tgagatctggatccgttcattgtacagcggtaaaaatcgggagtctgtcctacgaattcagctg

SEQ ID NO: 5 - DNA, 65 bp, Artificial Sequence
  random oligonucleotide
tgagatctggatccgttcatgcggtaaataagtccatcggaacgtgtgtcctacgaattcagctg

SEQ ID NO: 6 - DNA, 62 bp, Artificial Sequence
  random oligonucleotide
tgagatctggatccgttcggtaaaaatgagcaggggatcgaaatgtcctacgaattcagctg

SEQ ID NO: 7 - DNA, 65 bp, Artificial Sequence
  random oligonucleotide
tgagatctggatccgttcaaacagtgaaatggggcacggtagaactagtcctacgaattcagctg

SEQ ID NO: 8 - DNA, 64 bp, Artificial Sequence
  random oligonucleotide
tgagatctggatccgttcagaatagaaagaggacggttaaaaactagtcctacgaattcagctg

SEQ ID NO: 9 - DNA, 66 bp, Artificial Sequence
  synthetic oligonucleotide
tgagatctggatccgttcnnnnnnnnnnnnnnnnnnnnnnnnnnnnnngtcctacgaattcagctg

SEQ ID NO: 10 - DNA, 18 bp, Artificial Sequence
  primer with BamHI site
tgagatctggatccgttc

SEQ ID NO: 11 - DNA, 18 bp, Artificial Sequence
  primer with EcoR1 site
cagctgaattcgtaggac

SEQ ID NO: 12 - DNA, 18 bp, Artificial Sequence
  primer
gaacggatccagatctca

SEQ ID NO: 13 - DNA, 18 bp, Artificial Sequence
  primer
gtcctacgaattcagctg

SEQ ID NO: 14 - DNA, 65 bp, Artificial Sequence
  synthetic sequences flanking a random oligonucleotide
tgagatctggatccgttcgagcagtaaaagtaagaaaggcccgtttcgtcctacgaattcagctg

SEQ ID NO: 15 - DNA, 66 bp, Artificial Sequence
  synthetic sequences flanking a random oligonucleotide
tgagatctggaaccgttcggggaagggaaggtgaaagcaagaattaccgtcctacgaattcagctg

SEQ ID NO: 16 - DNA, 66 bp, Artificial Sequence
  synthetic sequences flanking a random oligonucleotide
tgagatctggattcgttcggggaagggaaggtgaaagcaagaattaccgtcctacgaattcagctg

SEQ ID NO: 17 - DNA, 66 bp, Artificial Sequence; identical to SEQ ID NO: 1
  synthetic sequences flanking a random oligonucleotide

SEQ ID NO: 18 - DNA, 66 bp, Artificial Sequence; identical to SEQ ID NO: 1
  synthetic sequences flanking a random oligonucleotide

SEQ ID NO: 19 - DNA, 66 bp, Artificial Sequence
  synthetic sequences flanking a random oligonucleotide
tgagatctggatcngttcggggaagggaaggtgaaagcaagaattaccgtcctacgaattcagctg

SEQ ID NO: 20 - DNA, 66 bp, Artificial Sequence
  synthetic sequences flanking a random oligonucleotide
tgagatctggatccgttcggggaagggaaggtgaaagcaagaattactgtcctacgaattcagctg

SEQ ID NO: 21 - DNA, 66 bp, Artificial Sequence
  synthetic sequences flanking a random oligonucleotide
ngagatctggatccgttcggggaagggaaggtgaaagcaaaaattaccgtcctacgaattcagctg

SEQ ID NO: 22 - DNA, 66 bp, Artificial Sequence
  synthetic sequences flanking a random oligonucleotide
ngagatctggatccgttcggggaagggaaggtgaaagtaagaattaccgtcctacgaatccagctg

SEQ ID NO: 23 - DNA, 66 bp, Artificial Sequence
  synthetic sequences flanking a random oligonucleotide
tgagatctggatccgttcggagaagggaaggtgaaggcaggaaataccgtcctacgaattcagctg

SEQ ID NO: 24 - DNA, 66 bp, Artificial Sequence; identical to SEQ ID NO: 2
  synthetic sequences flanking a random oligonucleotide

(19) United States
(12) Patent Application Publication
(10) Pub. No.: US 2001/0047525 A1
(43) Pub. Date: Nov. 29, 2001

(54) Novel root-preferred promoter elements and methods of use

(76) Inventors: Wesley B. Bruce, Urbandale, IA (US); Xiping Niu, Johnston, IA (US)

Correspondence Address: PIONEER HI-BRED INTERNATIONAL INC., 7100 N.W. 62ND AVENUE, P.O. BOX 1000, JOHNSTON, IA, 50131, US

(21) Appl. No.: 09/766,113

(22) Filed: Jan. 19, 2001

Related U.S. Application Data
(60) Provisional application No. 60/177,473, filed on Jan. 21, 2000.

(51) Int. Cl. A01H 5/00; C07H 21/04; C12N 5/14; C12N 15/82

(52) U.S. Cl. 800/298; 536/24.1; 435/320.1; 435/419; 800/278

(57) ABSTRACT

The present invention provides compositions and methods for regulating expression of nucleotide sequences in a plant. Compositions are novel nucleotide sequences for root-preferred promoter elements and plant promoters comprising the elements. Methods for expressing a nucleotide sequence in a plant using the promoter sequences disclosed herein are provided. The methods comprise transforming a plant cell with a nucleotide sequence operably linked to the promoters of the present invention and regenerating a stably transformed plant from the transformed plant cell.

### CROSS-REFERENCE TO RELATED APPLICATION

This application claims the benefit of, and hereby incorporates by reference, U.S. provisional patent application 60/177,473, filed Jan. 21, 2000.

### FIELD OF THE INVENTION

The present invention relates to the field of plant molecular biology, more particularly to regulation of gene expression in plants.

### BACKGROUND OF THE INVENTION

Expression of DNA sequences in a plant host is dependent upon the presence of an operably linked promoter that is functional within the plant host. Choice of the promoter sequence will determine when and where within the organism the DNA sequence is expressed. Thus, where continuous expression is desired throughout the cells of a plant, constitutive promoters are utilized. In contrast, where gene expression in response to a stimulus is desired, inducible promoters are the regulatory element of choice. Where expression in particular tissues or organs is desired, tissue-preferred promoters are utilized. In addition to the core promoter, regulatory sequences or promoter elements upstream and/or downstream from the core promoter sequence may be included in expression constructs of transformation vectors to bring about varying levels of expression of nucleotide sequences of interest in a transgenic plant.

Frequently it is desirable to have tissue-preferred expression of a DNA sequence in particular tissues or organs of a plant. For example, increased resistance of a plant to infection by soil- and air-borne pathogens might be accomplished by genetic manipulation of the plant's genome to comprise a tissue-preferred promoter operably linked to a pathogen-resistance gene such that pathogen-resistance proteins are expressed in the desired plant tissue. Alternatively, it might be desirable to inhibit expression of a native DNA sequence within a plant's tissues to achieve a desired phenotype.

A number of labs have identified promoter elements and corresponding DNA-binding proteins that are limited to specific tissues within the plant (See, e.g., Weising, et al Z. Naturforsch C46: 1; Oeda, et al EMBO J. 10:1793; Takatsuji, et al EMBO J 11:241; Yanagisawa, et al Plant Mol. Biol. 19:545; Zhou, et al J. Biol. Chem. 267:23515; Consonni, et al Plant J. 3:335; Foley, et al Plant J. 3:669; Matsuoka et al Proc. Natl. Acad. Sci. 90:9586). It is likely that a large number of DNA-binding factors will be limited to specific tissues, environmental conditions or developmental stages. It is considered important by those skilled in the art to develop transcriptional regulatory units that restrict gene expression to certain tissues of a plant. The ability to drive tissue-specific gene expression in plants is considered to be of agronomic importance to those skilled in the art.

Thus far, the regulation of gene expression in plant roots has not been extensively studied despite the root's importance to plant development. To some degree that is attributable to a lack of readily-available, root-specific biochemical functions whose genes may be cloned, studied, and manipulated. Genetically altering plants through the use of genetic engineering techniques and thus producing a plant with useful traits requires the availability of a variety of promoters. An accumulation of promoters would enable the investigator to design recombinant DNA molecules that are capable of being expressed at desired levels and cellular locales. Therefore, a collection of tissue-preferred promoters would allow for a new trait to be expressed in the desired tissue.

A promoter element or elements that specifically confer root-preferred expression has not been described. Short elements that may contribute to root-preferred expression have been disclosed; however, identification of the specific sequences responsible for root-specific gene expression have not been reported (Lam et al. (1989) Proc. Natl. Acad. Sci. USA 86:7890; see also Oliphant et al (1989) Mol. Cell Biol. 9: 2944-2949; Niu and Guiltinan (1994) Nucleic Acid Res. 22:4969-497; Oeda, et al EMBO J. 10:1793; and Catron et al. (1993) Mol. Cell Biol. 13: 2354-2365.)

Thus, methods and compositions directed to identification, isolation and characterization of promoters and promoter elements that can serve as regulatory regions for root-preferred expression of nucleotide sequences of interest are needed for genetic manipulation of plants.

### SUMMARY OF THE INVENTION

Compositions and methods for regulating expression of nucleotide sequences in a plant are provided. The compositions comprise novel nucleotide sequences for tissue-preferred, particularly root-preferred promoter elements (RPEs) and transcription regulatory units comprising the promoter elements. More particularly, plant promoters comprising one or more RPEs that enhance or suppress expression directed by the promoter are provided.

Methods for identifying and isolating tissue-preferred plant promoter elements are provided.

Methods for expressing a nucleotide sequence in a plant using the promoter sequences disclosed herein are also provided. The methods comprise transforming a plant cell with a transformation vector that comprises a nucleotide sequence operably linked to one of the plant promoters of the present invention and regenerating a stably transformed plant from the transformed plant cell. In this manner, expression levels in a plant cell, plant organ, plant tissue or plant seed can be controlled.

Transformed plants, seeds and plant cells comprising the transcription regulatory units and the promoter elements are also provided.

### BRIEF DESCRIPTION OF THE DRAWINGS

FIG. 1 depicts selected sequences for oligonucleotides from Random Oligonucleotide Library (ROL).

FIG. 2 depicts results of transient assay for CRC expression in roots, with constructs comprising selected ROL sequences.

FIG. 3 depicts results of transient assay for CRC expression in shoots, with constructs comprising selected ROL sequences.

### DETAILED DESCRIPTION OF THE INVENTION

Compositions of the present invention are directed to novel nucleotide sequences for tissue-preferred, particularly for root-preferred promoter elements (RPEs) and plant promoters comprising the promoter elements. The promoter elements of the invention can be used in combination with a promoter or transcription regulatory region to direct expression in particular tissues and to modulate levels of transcription of an operably linked nucleotide sequence. That is, the promoter elements are useful for enhancing or suppressing expression of an operably linked sequence.

As used herein, the term “plant” includes reference to whole plants and their progeny; plant cells; plant parts or organs, such as embryos, pollen, ovules, seeds, flowers, kernels, ears, cobs, leaves, husks, stalks, stems, roots, root tips, anthers, silk and the like. Plant cell, as used herein, further includes, without limitation, cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. The class of plants which can be used in the methods of the invention is generally as broad as the class of higher plants amenable to transformation techniques, including both monocotyledonous and dicotyledonous plants. A particularly preferred plant is Zea mays.

By “tissue-preferred” is intended that the expression driven by a plant promoter is selectively enhanced or suppressed in particular plant cells or tissues, in comparison to other cells or tissues.

By “root-preferred” is intended that the expression driven by a plant promoter is selectively enhanced or suppressed in root cells or tissues, in comparison to one or more non-root cells or tissues. Root tissues include but are not limited to at least one of root cap, apical meristem, protoderm, ground meristem, procambium, endodermis, cortex, vascular cortex, epidermis, and the like. Roots include primary, lateral and adventitious roots.

By “root-preferred promoter element” or “RPE” is intended a promoter element that enhances or suppresses expression driven by a promoter in a plant cell in a root-preferred manner.

By “promoter” or “transcriptional initiation region” is intended a regulatory region of DNA usually comprising a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, and referred to as “promoter elements” which influence the expression driven by the core promoter. Promoter elements located upstream or 5′ to the TATA box are also referred to as upstream promoter elements. In particular embodiments of the invention, the promoter elements of the invention are positioned upstream or 5′ to the TATA box. However, the invention also encompasses plant promoter configurations in which the promoter elements are positioned downstream or 3′ to the TATA box.

By “transcription regulatory unit” is intended a promoter comprising one or more promoter elements.

By “core promoter” is intended a promoter not comprising promoter elements other than the TATA box and the transcriptional start site.

The transcription regulatory units of the invention comprising the RPEs, when operably linked to a nucleotide sequence of interest and inserted into a transformation vector, control root-preferred expression of the linked nucleotide sequence in the cells of a plant stably transformed with this vector. That is, the expression of this linked sequence is enhanced or suppressed in root cells or tissues in comparison to one or more non-root cells or tissues, and in comparison to non-transformed cells or tissues.

While the linked nucleotide sequence of interest is heterologous to the promoter element sequence, it may be native or foreign to the plant host. The invention encompasses expression of native coding sequences, particularly the coding sequences related to pathogen-resistance phenotype, linked to a promoter of the invention. The use of the promoter elements to express the native coding sequences will alter the phenotype of the transformed plant or plant cell.

The promoter elements of the invention may be used with any promoter, particularly plant promoters. Such promoters may be native or synthetic. By “plant promoter” is intended a promoter capable of driving expression in a plant cell.

In reference to a promoter, by “native” is intended a promoter capable of driving expression in a cell of interest, wherein the nucleotide sequence of the promoter is found in the cell of interest in nature.

In reference to a promoter or transcription initiation region, by “synthetic” is intended a promoter capable of driving expression in a cell of interest, wherein the nucleotide sequence of the promoter is not found in nature. A synthetic promoter cannot be isolated from any cell unless it is first introduced to the cell or to an ancestor thereof.

The invention encompasses isolated or substantially purified nucleic acid compositions comprising novel combinations of promoter elements, and transcription regulatory units with promoter elements. Particularly, the nucleotide sequences for RPEs are provided including, RPE 15 (SEQ ID NO.: 1), RPE14 (SEQ ID NO.: 2), RPE19 (SEQ ID NO.: 3), RPE29 (SEQ ID NO.: 4), RPE60 (SEQ ID NO.: 5), RPE2 (SEQ ID NO.: 6), RPE 39 (SEQ ID NO.: 7) and RPE 61 (SEQ ID NO.: 8). An “isolated” or “purified” nucleic acid molecule, or biologically active portion thereof, is substantially free of other cellular material, or culture medium when produced by recombinant techniques, or substantially free of chemical precursors or other chemicals when chemically synthesized.

The promoter elements of the invention may act as enhancers or suppressors of expression. That is, the promoter elements of the invention can enhance or suppress expression of nucleotide sequences operably linked to the plant promoters comprising the promoter elements depending on the choice of the particular promoter, the particular construct and the host cell.

Enhancers are nucleotide sequences that act to enhance or increase the expression directed by a promoter region. This increase or enhancement can be determined by comparing the expression level directed by a sample promoter comprising a putative enhancer placed at any position upstream or downstream of the promoter, relative to a control promoter that does not comprise a putative enhancer. Enhancer elements for plants are known in the art and include, for example, the SV40 enhancer region, the 35S enhancer element, and the like. Particular examples of RPEs of the invention that act as enhancers include RPE14 (SEQ ID NO.: 2), RPE19 (SEQ ID NO.: 3), RPE29 (SEQ ID NO.: 4), RPE60 (SEQ ID NO.: 5), RPE2 (SEQ ID NO.: 6), and RPE 61 (SEQ ID NO.: 8), as described in Example 3 below.

By “suppressors” are intended nucleotide sequences that mediate suppression or decrease in the expression directed by a promoter region. That is, suppressors are the DNA sites through which transcription repressor proteins exert their effects. Suppressors can mediate suppression of expression by overlapping transcription start sites or transcription activator sites, or they can mediate suppression from distinct locations with respect to these sites. Particular examples of an RPE of the invention that acts as a suppressor includes RPE15 (SEQ ID NO.: 1) and RPE39 (SEQ ID NO.: 7).

The invention encompasses multimeric RPEs. By “multimeric RPE” is intended herein a promoter element comprising a first copy of an RPE of the present invention, or a fragment or variant thereof; and at least a second copy of an RPE of the present invention, or a fragment or variant thereof. The invention also encompasses promoters comprising the multimeric RPEs. The multimeric RPEs include but are not limited to those comprising two or more copies of the same RPE; those comprising one or more copies of at least two different RPEs; and any combination of fragments and variants thereof. In this aspect of the present invention, each individual RPE could be in the antisense orientation. By “orientation” is intended the 5′ to 3′ (sense) or the 3′ to 5′ (antisense) configuration of a promoter element sequence contained in a contiguous strand, relative to the configuration of other promoter elements and/or the TATA box contained in that strand.

The invention encompasses multimeric RPEs in which the individual promoter elements of the invention are separated and/or flanked by spacer sequences. By “spacer sequence” is intended the nucleotide sequence contained in a multimeric RPE that is not a promoter element sequence. The invention also encompasses multimeric RPEs comprising contiguous multimers of individual promoter elements, thereby containing no spacer sequences; multimeric RPEs in which one or more individual elements are separated or flanked by spacer sequences; and multimeric RPEs comprising spacer sequences that are different than the spacer sequences disclosed herein.

The RPEs may be operably linked to any promoter of interest. While not a limitation, it may be preferable to use core promoters. Promoters, particularly core promoters of interest, may be derived from a variety of sources.

Constitutive promoters include, for example, the core promoter of the Rsyn7 (U.S. Pat. No. 6,072,050); the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol 12:619-632 and Christensen et al. (1992) Plant Mol. Biol. 18:675-689); pEMU (Last et al. (1991) Theor. Appl Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; and 5,608,142.

The isolated RPE sequences of the present invention, and plant promoter sequences comprising the RPEs, can be modified to provide for a range of expression levels of the nucleotide sequence of interest. Thus, less than the entire promoter regions may be utilized and the ability to drive expression of the coding sequence retained. However, it is recognized that expression levels of the mRNA may be decreased with deletions of portions of the promoter sequences. Likewise, the general nature of expression may be changed.

Modifications of the promoter element sequences of the present invention and of plant promoter sequences comprising the promoter elements can provide for a range of expression. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

The nucleotide sequences for the plant promoters of the present invention may comprise the sequences set forth in SEQ ID NOS: 1-8 or any sequence having substantial identity to the sequences. By “substantial identity” is intended a sequence exhibiting substantial functional and structural equivalence with the sequence set forth. Any functional or structural differences between substantially identical sequences do not affect the ability of the sequence to function as a promoter as disclosed in the present invention. Thus, the plant promoter of the present invention will direct enhanced or repressed root-preferred expression of an operably linked nucleotide sequence. Two RPE nucleotide sequences are considered substantially identical when they have at least about 80%, preferably at least about 85%, more preferably at least about 90%, still more preferably at least about 95%, and most preferably at least about 98% sequence identity.

Fragments and variants of the RPE nucleotide sequences set forth herein are encompassed by the present invention. Promoters comprising biologically active fragments of the RPEs of the invention are also encompassed by the present invention. By “fragment” is intended a portion of the promoter element nucleotide sequence that is shorter than the full-length promoter element sequence. Fragments of a nucleotide sequence may retain biological activity and hence enhance or suppress expression of a nucleotide sequence operably linked to a promoter comprising the promoter element fragment. Alternatively, fragments of a nucleotide sequence that are useful as hybridization probes or PCR primers generally do not retain biological activity. Thus, fragments of a nucleotide sequence may range from at least about 15, 20, or 25 nucleotides, and up to but not including the full-length of a nucleotide sequence of the invention.

A biologically active portion of a promoter comprising the promoter element fragment of the invention can be prepared by synthesizing a promoter comprising a portion of one of the RPE sequences and assessing the activity of the fragment.

The invention encompasses variants of the RPEs and of plant promoters comprising the RPEs. By “variants” is intended substantially identical sequences. Naturally-occurring variants of the promoter element sequences can be identified and/or isolated with the use of well-known molecular biology techniques, as, for example, with PCR and hybridization techniques as outlined below. The invention encompasses variants of the RPEs and plant promoter sequences disclosed herein in which the promoter elements of the invention are substituted by a natural variant of that element.

Variants also encompass synthetically derived nucleotide sequences, such as those generated by using site-directed mutagenesis or automated oligonucleotide synthesis. Methods for mutagenesis and nucleotide sequence alterations are well known in the art. Generally, variants of the RPE nucleotide sequences of the invention will have at least 80%, preferably 85%, 90%, 95%, up to 98% or more sequence identity to an RPE nucleotide sequence of the invention.

Biologically active variants of the promoter element sequences should retain promoter regulatory activity, and thus enhance or suppress expression of a nucleotide sequence operably linked to a transcription regulatory unit comprising the promoter element. Promoter activity may be measured by Northern blot analysis. See, for example, Sambrook et al. (1989) Molecular Cloning: A Laboratory Manual (2d ed., Cold Spring Harbor Laboratory Press, Plainview, N.Y.); herein incorporated by reference. Protein expression indicative of promoter activity can be measured by determining the activity of a protein encoded by the coding sequence operably linked to the particular promoter; including but not limited to such examples as GUS (b-glucoronidase; Jefferson (1987) Plant Mol. Biol. Rep. 5:387), GFP (green florescence protein; Chalfie et al. (1994) Science 263:802), luciferase (Riggs et al. (1987) Nucleic Acids Res. 15(19):8115 and Luehrsen et al. (1992) Methods Enzymol. 216:397-414), and the maize genes encoding for anthocyanin production (Ludwig et al. (1990) Science 247:449).

The invention also encompasses nucleotide sequences which hybridize to the promoter element sequences of the invention under stringent conditions, and enhance or suppress expression of a nucleotide sequence operably linked to a transcription regulatory unit comprising the promoter element. Hybridization methods are known in the art. See, for example Sambrook et al. (1989) Molecular Cloning: A Laboratory Manual (2d ed., Cold Spring Harbor Laboratory Press, Plainview, N.Y.). See also Innis et al., eds. (1990) PCT Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of identity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2× SSC (20× SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1× SSC at 55 to 60° C. Exemplary high stringency conditions include hybridization in 50% formamide, 1 M NaCl, 1% SDS at 37° C., and a wash in 0.1× SSC at 60 to 65° C. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (%GC)−0.61 (% form)−500/L; where M is the molarity of monovalent cations, %GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

In general, the invention comprises sequences which are at least about 80% homologous to the sequences disclosed herein and have promoter or enhancer activity.

To determine extent of identity of two sequences, methods of alignment are well known in the art. Thus, the determination of percent identity between any two sequences can be accomplished using a mathematical algorithm. Preferred, non-limiting examples of such mathematical algorithms are the algorithm of Myers and Miller (1988) CABIOS 4:11 -17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

For purposes of the present invention, comparison of nucleotide or protein sequences for determination of percent sequence identity to the sequences disclosed herein is preferably made using the BLASTN program (BLAST Version 2.0 or later) with its default parameters or any equivalent sequence comparison program. By “equivalent program” is intended any sequence comparison program that, for any two sequences in question, generates an alignment having identical nucleotide or amino acid residue matches and an identical percent sequence identity when compared to the corresponding alignment generated by the preferred program.

As used herein, “sequence identity” or “identity” in the context of two nucleic acid sequences makes reference to the residues in the two sequences that are the same when aligned for maximum correspondence over a specified comparison window.

The term “substantial identity” of polynucleotide sequences means that a polynucleotide comprises a sequence that has at least 80% sequence identity, preferably at least 85%, more preferably at least 90%, even more preferably at least 95%, and most preferably at least 98%, compared to a sequence of the invention using one of the alignment programs described above using standard or default parameters.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein.

The invention provides methods for identifying and isolating tissue-preferred plant promoter elements, including but not limited to the root-preferred promoter elements. The identification and isolation methods of the invention are directed to construction and use of random oligonucleotide libraries (ROLs), binding the oligonucleotides with proteins from crude nuclear extracts from a plant tissue of interest, separating and isolating the bound complexes on electrophoretic mobility shift assay gels (EMSA), amplifying the bound oligonucleotides from specific electrophoretic mobility ranges; repeating the cycle of binding, separating, isolating, amplifying; and comparing the quantity of bound complex formation in progressive cycles for a particular electrophoretic mobility range. In this manner, where a particular range exhibits increased complex formation in progressive cycles, that range is assessed to comprise desired tissue-preferred promoters. Individual oligonucleotides can be isolated from this enriched population by cloning, operably linked with a promoter, and assessed for enhancement or repression of expression directed by the promoter. Those oligonucleotides capable of enhancing or repressing the expression in a tissue-preferred manner are identified as tissue-preferred promoters, and their sequence determined.

Known approaches of identifying and isolating tissue-specific promoter elements from promoter sequences are generally labor intensive. Such approaches usually begin with identifying the genes that are differentially regulated using either, for example, differential or subtractive cDNA library screening methods or PCR-based differential display. See Liang and Pardee (1992) Science 257: 967-971; Sharma and Davis (1995) Plant Mol. Biol. 29: 91-98. These approaches are typically followed by the cloning of genomic 5′-flanking sequences corresponding to the desired cDNA and ending with an exhaustive dissection of the flanking sequences via either interaction with trans-acting factors or functional expression assays.

In other known methods, the DNA-binding sites of known trans-acting factors are determined using reiterative binding enrichment methods with random oligonucleotide libraries. See, for example, Catron et al. (1993) Mol. Cell Biol. 13: 2354-2365; Ko and Engel (1993) Mol. Cell Biol. 13: 4011-4022; Niu and Guiltinan (1994) Nucleic Acid Res. 22: 4969-497; Norby et al. (1992) Nucleic Acids Res. 20: 6317-6321; Oliphant et al (1989) Mol. Cell Biol. 9: 2944-2949.

These approaches depend on the availability of either purified DNA-binding factors or antibodies directed to the DNA-binding factors. Nallur et al. (1996) describes a multiplex selection technique (MuST) that enriches for transcription factor binding sites from an ROL using crude nuclear extracts. WO97/44448 describes utilization of ROLs and nuclear extracts for generating tissue-preferred libraries of promoter elements, by selecting for elements that bind nuclear extracts from a preferred tissue, but not other tissues. Others have exploited massive parallel approaches of expression profiling and entire genome sequencing to identify cis-elements, common to the coordinately controlled genes. See Roth et al. (1998) Nature Biotech. 16: 939-945.

The isolation and identification methods of the present invention are not dependent on genomic sequences, prior knowledge of particular trans-acting factors, or availability of purified DNA-binding factors or antibodies directed to the DNA-binding factors for identification and isolation of tissue-preferred promoter elements. Furthermore, because particular populations of sequences are enriched in the course of isolation and identification, subsequent cloning and expression analysis is much less laborious and extensive.

The nucleotide sequences for the RPEs and promoters of the present invention, as well as variants and fragments thereof, are useful in the genetic manipulation of any plant when operably linked with a nucleotide sequence whose expression is to be controlled to achieve a desired phenotypic response. By “operably linked” is intended that the transcription or translation of the nucleotide sequence of interest is under the influence of the promoter sequence. In this manner, the nucleotide sequences for the promoters of the invention are provided in expression cassettes along with nucleotide sequences of interest for expression in the plant of interest.

Such nucleotide constructs or expression cassettes will comprise a transcriptional initiation region in combination with a promoter element operably linked to the nucleotide sequence whose expression is to be controlled by the promoters disclosed herein. Such construct is provided with a plurality of restriction sites for insertion of the nucleotide sequence to be under the transcriptional regulation of the regulatory regions. The expression cassette may additionally contain selectable marker genes.

The transcriptional cassette will include in the 5′-to-3′ direction of transcription, a transcriptional and translational initiation region, one or more promoter elements, a nucleotide sequence of interest, and a transcriptional and translational termination region functional in plant cells. The termination region may be native with the transcriptional initiation region comprising one or more of the promoter nucleotide sequences of the present invention, may be native with the DNA sequence of interest, or may be derived from another source. Convenient termination regions are available from the Ti-plasmid of A. tumefaciens, such as the octopine synthase and nopaline synthase termination regions. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. 1989) Nucleic Acids Res. 17:7891-7903; Joshi et al. (1987) Nucleic Acid Res. 15:9627-9639.

The expression cassette comprising the transcription regulatory unit of the invention operably linked to a nucleotide sequence may also contain at least one additional nucleotide sequence for a gene to be cotransformed into the organism. Alternatively, the additional sequence(s) can be provided on another expression cassette.

Where appropriate, the nucleotide sequence whose expression is to be under the control of the promoter sequence of the present invention, and any additional nucleotide sequence(s), may be optimized for increased expression in the transformed plant. That is, these nucleotide sequences can be synthesized using plant-preferred codons for improved expression. Methods are available in the art for synthesizing plant-preferred nucleotide sequences. See, for example, U.S. Pat. Nos. 5,380,831 and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the nucleotide sequence of interest may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Nat. Acad. Sci. USA 86:6126-6130); potyvirus leaders, for example, TEV leader (Tobacco Etch Virus) (Allison et al. (1986)); MDMV leader (Maize Dwarf Mosaic Virus) (Virology 154:9-20); human immunoglobulin heavy-chain binding protein (BiP) (Macejak and Sarnow (1991) Nature 353:90-94); untranslated leader from the coat protein mRNA of alfalfa mosaic virus (AMV RNA 4) (Jobling and Gehrke (1987) Nature 325:622-625); tobacco mosaic virus leader (TMV) (Gallie et al. (1989) Molecular Biology of RNA, pages 237-256); and maize chlorotic mottle virus leader (MCMV) (Lommel et al. (1991) Virology 81:382-385). See also Della-Cioppa et al. (1987) Plant Physiology 84:965-968. Other methods known to enhance translation and/or mRNA stability can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, substitutions, for example, transitions and transversions, may be involved.

The promoters may be used to drive reporter genes or selectable marker genes. Examples of suitable reporter genes known in the art can be found in, for example, Jefferson et al. (1991) in Plant Molecular Biology Manual, ed. Gelvin et al. (Kluwer Academic Publishers), pp. 1-33; DeWet et al. (1987) Mol. Cell. Biol. 7:725-737; Goff et al. (1990) EMBO J. 9:2517-2522; and Kain et al. (1995) BioTechniques 19:650-655; and Chiu et al. (1996) Current Biology 6:325-330.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella et al. (1983) EMBO J. 2:987-992); methotrexate (Herrera Estrella et al. (1983) Nature 303:209-213; Meijer et al. (1991) Plant Mol. Biol. 16:807-820); hygromycin (Waldron et al. (1985) Plant Mol. Biol. 5:103-108; Zhijian et al. (1995) Plant Science 108:219-227); streptomycin (Jones et al. (1987) Mol. Gen. Genet. 210:86-91); spectinomycin (Bretagne-Sagnard et al. (1996) Transgenic Res. 5:131-137); bleomycin (Hille et al. (1990) Plant Mol. Biol. 7:171-176); sufonamide (Guerineau et al. (1990) Plant Mol. Biol. 15:127-136); bromoxynil (Stalker et al. (1988) Science 242:419-423); glyphosate (Shaw et al. (1986) Science 233:478-481); phosphinothricin (DeBlock et al. (1987) EMBO J 6:2513-2518).

Other genes that could serve utility in the recovery of transgenic events but might not be required in the final product would include, but are not limited to, such examples as GUS (b-glucoronidase; Jefferson (1987) Plant Mol. Biol. Rep. 5:387), GFP (green fluorescence protein; Chalfie et al. (1994) Science 263:802), luciferase (Riggs et al. (1987) Nucleic Acids Res.15(19):8115 and Luehrsen et al. (1992) Methods Enszymol. 216:397-414), and the maize genes encoding for anthocyanin production (Ludwig et al. (1990) Science 247:449).

The expression cassette comprising the transcription regulatory unit of the present invention operably linked to a nucleotide sequence of interest can be used to transform any plant. In this manner, genetically modified plants, plant cells, plant tissue, seed, and the like can be obtained. Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055), direct gene transfer (Paszkowski et al. (1984) EMBO J. 3:2717-2722), and ballistic particle acceleration (see, for example, Sanford et al., U.S. Pat. No. 4,945,050; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol 91:440-444 (maize); Fromm et al. (1990) Biotechnology 8:833-839 (maize); Hooykaas-Van Slogteren et al. (1984) Nature (London) 311:763-764; Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In certain preferred embodiments in this regard, the vectors provide for preferred expression. Such preferred expression may be inducible expression or temporally limited or restricted to predominantly certain types of cells or any combination of the above. Particularly preferred among inducible vectors are vectors that can be induced for expression by environmental factors that are easy to manipulate, such as temperature and nutrient additives. A variety of vectors suitable to this aspect of the invention, including constitutive and inducible expression vectors for use in prokaryotic and eukaryotic hosts, are well known and employed routinely by those of skill in the art. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids and binaries used for Agrobacterium-mediated transformations. All may be used for expression in accordance with this aspect of the present invention.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

The present invention may be used for transformation of any plant species, including, but not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Preferably, plants of the present invention are crop plants (for example, corn, alfalfa, sunflower, Brassica, soybean, cotton, safflower, peanut, sorghum, wheat, millet, tobacco, etc.), more preferably corn and soybean plants, yet more preferably corn plants.

Plants of particular interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The promoter sequences and methods disclosed herein are useful in regulating expression of a nucleotide sequence of interest in a host plant in a tissue-preferred manner, more particularly in a root-preferred manner. Thus, the nucleotide sequence operably linked to the promoters disclosed herein may be a structural gene encoding a protein of interest. Examples of such genes include, but are not limited to, genes encoding proteins conferring resistance to abiotic stress, such as drought, temperature, salinity, and toxins such as pesticides and herbicides, or to biotic stress, such as attacks by fungi, viruses, bacteria, insects, and nematodes, and development of diseases associated with these organisms.

Alternatively, the nucleotide sequence operably linked to one of the promoters disclosed herein may be an antisense sequence for a targeted gene. Thus, sequences can be constructed which are complementary to, and will hybridize with, the messenger RNA (mRNA) of the targeted gene. Modifications of the antisense sequences may be made, as long as the sequences hybridize to and interfere with expression of the corresponding mRNA. In this manner, antisense constructions having 70%, preferably 80%, more preferably 85% sequence similarity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used. When delivered into a plant cell, expression of the antisense DNA sequence prevents normal expression of the DNA nucleotide sequence for the targeted gene. In this manner, production of the native protein encoded by the targeted gene is inhibited to achieve a desired phenotypic response. Thus the promoter is linked to antisense DNA sequences to reduce or inhibit expression of a native protein in the plant.

In a preferred embodiment of the present invention, the root-preferred promoters and/or promoter elements are used to enhance or suppress expression of nucleotide sequences encoding proteins directly involved in agronomically important traits in root, or those encoding root proteins that affect agronomically important traits in non-root tissue.

It is recognized that tissue-preferred promoter elements identified and isolated according to the methods of the present invention can be used to enhance or suppress expression of agronomically important traits in a tissue-preferred manner.

The following examples are offered by way of illustration and not by way of limitation.

### EXPERIMENTAL

### EXAMPLE 1

Isolation and Identification of RPEs

Design and Preparation of Random Oligo Library (ROL):

A Random Oligo Library (ROL) was designed and constructed to have about 30 nucleotides of randomized sequence. The complexity of the ROL is about 1.15e+18 unique molecules. Two considerations were taken into account in designing this ROL. First, transcription control may be determined by various transcriptional complexes including multiple transcription factors, co-activators, and other associated factors. The long random sequence in the ROL (about 30 nucleotides) allows selection of complex binding sequences by these multiple factors. Second, the promoter elements may be located at different positions along the random sequence. Therefore, spacing of the promoter elements can be tested in subsequent functional promoter analysis. In addition, the spacer sequences flanking the randomized sequences of the ROL were also carefully designed so that they do not contain known transcription factor binding sites. The ROL and flanking primers used to amplify ROL are shown below:

n19813 5′TGAGATCTGGATCCGTTC(N)30GTCCTACGAATTCAGCTG3′

n19808 5′TGAGATCTGGATCCGTTC3′

n19811 5′CAGCTGAATTCGTAGGAC3′

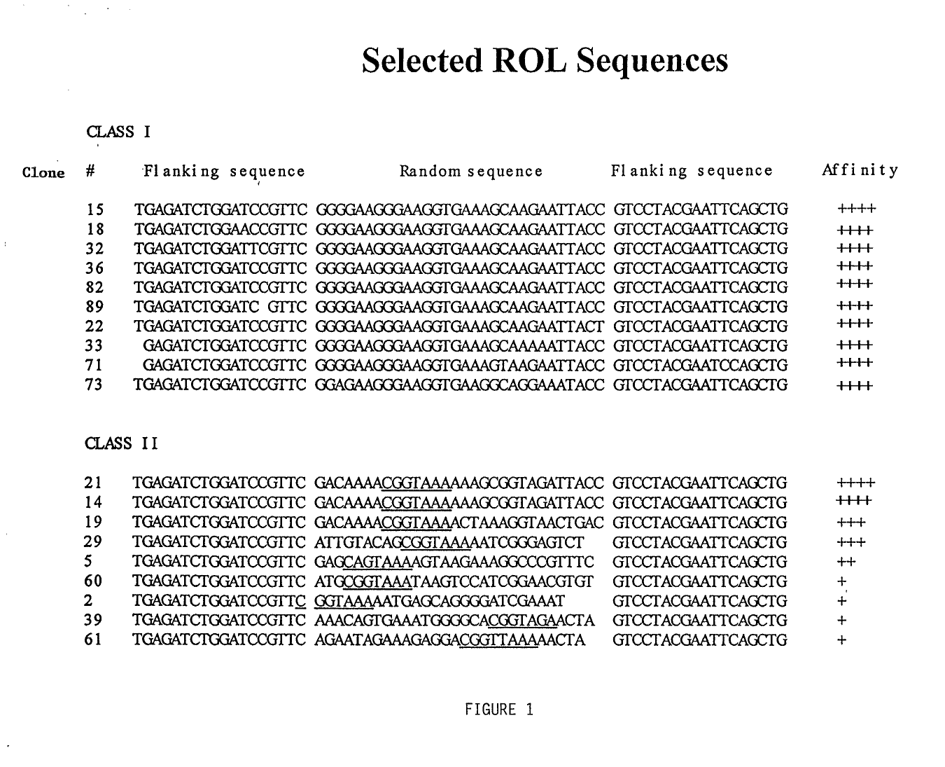

The basic structure of each oligonucleotide of the ROL is shown above as n19813 (SEQ ID NO: 9), wherein “(N)” designates the position of the random oligonucleotide sequence, relative to the 5′ and the 3′ flanking spacer sequences (see also FIG. 1). The ROL (n19813) was annealed to one primer (n19811) and labeled by Klenow enzyme in presence of α-P³²-dCTP using a standard protocol. The labeled probe was then gel purified before use in DNA binding reactions with maize nuclear proteins.

n19808 and n19811 designate primer pairs used for PCR amplification of the ROL, and contain BamHI and EcoRl sites respectively, for cloning purposes.

Maize Nuclear Protein Preparation:

Maize nuclear extracts were prepared using a protocol modified from Green et al. (1988) “In vitro DNA Footprinting,” in Plant Molecular Biology Manual, ed. Gelvin, Schilperoort, and Verma (Kluwer Academic Publishers, Dordrecht ) B11: 1-22. Briefly, maize inbred A63 seeds were germinated in the dark at 24° C. Roots from 4-day seedlings were collected and 4× volume of the Homogenizing Buffer (HB) (25 mM Hepes/KOH pH 7.6, 10 mM MgCl2, 0.3 M sucrose, 0.5% Triton X-100, 5 mM β-mercaptoethanol, 1 mM PMSF) was added. Tissues were dissected into small pieces using a commercial Waring blender at low speed for 10 seconds (sec.) and ground to paste with mortar and pestle. Homogenized tissues were filtered through two layers of miracloth (CalBiochem) and one layer of 70 μm nylon screen. The extracts were centrifuged in a Sorval GSA rotor at 4500 rpm, for 15 minutes (min.). Nuclei pellets were then resuspended gently with a paint brush in HB and centrifuged as above. This step was repeated once. After the last centrifugation, nuclei were resuspended in Nuclear Lysis Buffer (15 mM Hepes/KOH pH 7.6, 110 mM KCl, 5 mM MgCl2, 1 mM DTT, 1 mM PMSF, 5 ug/ml leupeptin, 2 ug/ml aprotinin, 1 ug/ml pepstatin A). NaCl was added in a drop-wise manner to a final concentration of 0.5 M. Nuclear proteins were extracted from the nuclei by incubation of the NaCl mixture on ice for 40 min. with gentle shaking. The extract was centrifuged in Sorval SS34 rotor, 16K rpm, for 30 min. Supernatants were frozen in liquid nitrogen and stored at −80° C. Frozen nuclear extracts were thawed on ice, and ammonium sulfate was added slowly to the nuclear extracts to a final concentration of 0.35 mg/ml while stirring. Precipitated nuclear proteins were centrifuged in a Sorval SS34 rotor at 16k rpm for 30 min. The pellets were resuspended in Nuclear Extract Buffer (NEB) (25 mM Hepes/KOH pH 7.6, 40 mM KCl, 0.1 mM EDTA, 10% glycerol, 5 mM β-mercaptoethanol) with 1 mM PMSF, 5 ug/ml antipain, 5 ug/ml leupeptin and 5 ug/ml aprotinin and dialyzed for 6 hours against NEB with 0.1 mM PMSF. The dialyzed nuclear extracts were aliquoted and stored at −80° C. until use.

DNA Binding Reactions and Selection Process:

For the DNA binding reactions, 1-5 μg nuclear extracts in NEB buffer were incubated on ice for 5-20 min. with the labeled ROL oligonucleotide in the presence of 1 μg poly(dI-dC), and 0.7 μg of two annealed primer pairs for ROL flanking sequences.

The primer pairs were:

Left=n19808 (sequence provided above) and n19809 (GAACGGATCCAGATCTCA)

Right=n19811 (sequence provided above) and n19810 (GTCCTACGAATTCAGCTG)

Binding reactions were run on native polyacrylamide gels according to a standard EMSA (electrophoresis mobility shift assay) protocol, basically as described in McKendree et al (1990) Plant Cell 2: 207-214. The gel area corresponding to the bound fractions was divided into 10 bands. DNA was eluted from each gel band, PCR amplified using primers n19808 (SEQ ID NO.: 10) and n19811 (SEQ ID NO.: 11) in the presence of α-P³²-dCTP, and gel purified. The resulting labeled-DNA probes were combined into three pools corresponding to high-, medium-, and low-molecular-weight bands. These three pools of probes were used separately for the next round of the selection process. Six rounds of selection were performed on the ROL with maize root nuclear extracts.

At the position of gel band 3 (a member of the medium-molecular-weight pool), a specific DNA-binding complex was detectable. Electrophoretic mobility shift assay (EMSA) showed that the binding sequences for this specific complex were progressively enriched during the selection process.

Cloning and Sequencing of the Selected ROL Oligonucleotides:

After six rounds of selection, the selected oligonucleotides in gel band 3 (high-molecular weight bands) were cloned into expression analysis vectors utilizing the SynCoreII core promoter or the Ubiquitin Core Promoter-Rsyn7, and CRC activator as a reporter system; described in U.S. Pat. No. 6,072,050, the contents of which are herein incorporated by reference. Nineteen clones were recovered and sequenced. Sequence alignment indicates that two classes of sequences were present in about equal proportions within the selected pool. The selected DNA sequences in the first class were almost identical. In the second class of selected DNA sequences, a major motif (ACGGTAAA) was present among these sequences. See FIG. 1.

### EXAMPLE 2

In vitro Binding Study of the Selected Oligonucleotides

To test whether the cloned sequences are true binding sites for the observed nuclear complex, and for relative affinity among these selected sequences, the 19 cloned sequences were labeled and tested for binding of root nuclear proteins. EMSA or bandshift assays were carried out basically as described in WO97/44448. The results indicated that all 19 selected oligonucleotides are more strongly bound by root nuclear proteins than are randomly-chosen sequences in the random oligonucleotide library (ROL). The sequences in Class I are all high-affinity binding sequences, but the affinities of the sequences in Class II vary (FIG. 1). High affinity is indicated by the fact that more DNA was bound by protein in nuclear extract as determined from the gel shift assays. Low affinity is indicated by the fact that less DNA was bound by protein in nuclear extract, the bound fraction being in lesser quantity than that corresponding to high affinity, as determined from the gel shift assays.

### EXAMPLE 3

Transient Assays of the Selected Oligonucleotides

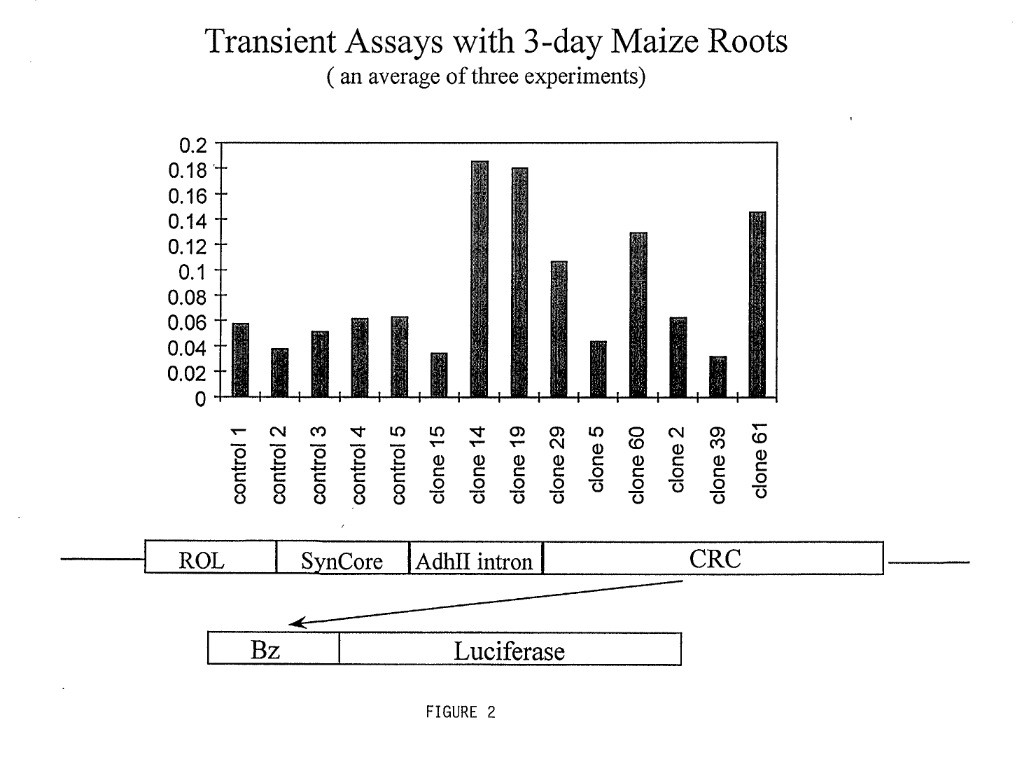

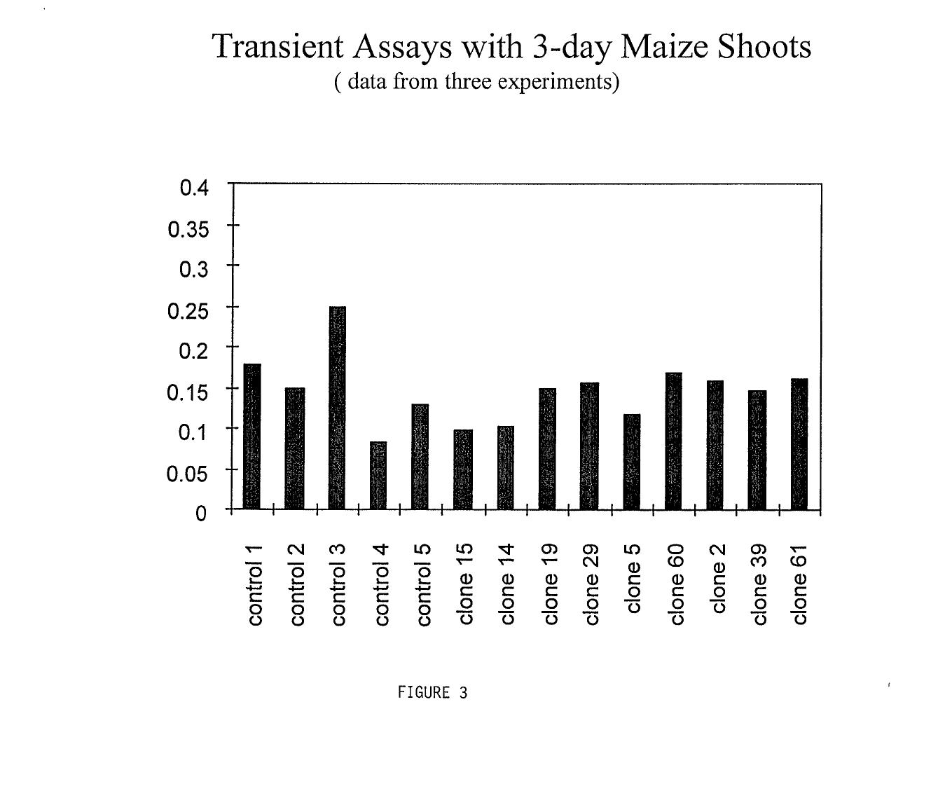

To functionally test the selected DNA sequences for their promoter activity in vivo, one sequence representing Class I and 8 sequences from Class II were chosen for transient assay by particle gun bombardment. Briefly, 3-day seedlings of the W22 R-g Stadler line were bombarded with 3 μg of the experimental plasmid (ROL oligonucleotide::Syncore::AdhII::CRC::PinII), 3 μg of reporter plasmid (Bz1L::LUC) and 1 μg internal standard (Ubi-Ubi::GUS). See U.S. Pat. No. 6,072,050 for basic constructs. See Tomes et al. (Tomes, D. et al., IN: Plant Cell, Tissue and Organ Culture: Fundamental Methods, Eds. O. L. Gamborg and G. C. Phillips, Chapter 8, pgs. 197-213 (1995)) for bombardment process. Following a 20-hour incubation in the dark, crude protein extracts were prepared from roots. 20 μl and 2 μl of tissue extracts were used for luciferase (LUC) and GUS activities, respectively. The promoter activity was expressed as a ratio of LUC activity over GUS activity. The transient assays indicated that all of the sequences in Class II can activate reporter gene expression in roots to some extent. Generally, the binding affinity for nuclear proteins correlated positively with promoter activity, except for the Class I sequences. See FIGS. 1 and 2. The transient assays also show that the selected sequences did not elevate reporter gene expression in shoots as compared to the controls (FIG. 3).

Based on these transient assays, promoter elements of SEQ ID NOS.: 2, 3, 4, 5, 6, and 8 were selected as enhancers for expression in root, and promoter elements of SEQ ID NOS.: 1 and 7 were selected as repressors for expression in root.

The isolated promoter elements have no exact match with any sequences in public databases. The sequence CGGTAA is present in the rice PhyA promoter, as described in Dehesh et al. (1990) Science 250:1397-1399. The promoter elements described herein can confer root-preferred gene expression. The root-preferred promoter elements are set forth in SEQ ID NOS.: 1-8.

### EXAMPLE 4

Transformation and Regeneration of Transgenic Maize

Biolistics:

The inventive polynucleotides contained within a vector are transformed into embryogenic maize callus by particle bombardment, generally as described by Tomes, D. et al., IN: Plant Cell, Tissue and Organ Culture: Fundamental Methods, Eds. O. L. Gamborg and G. C. Phillips, Chapter 8, pgs. 197-213 (1995) and as briefly outlined below. Transgenic maize plants are produced by bombardment of embryogenically responsive immature embryos with tungsten particles associated with DNA plasmids. The plasmids consist of a selectable marker gene and a structural gene of interest.

Preparation of Particles:

Fifteen mg of tungsten particles (General Electric), 0.5 to 1.8μ, preferably 1 to 1.8μ, and most preferably 1μ, are added to 2 ml of concentrated nitric acid. This suspension was sonicated at 0° C. for 20 minutes (Branson Sonifier Model 450, 40% output, constant duty cycle). Tungsten particles are pelleted by centrifugation at 10000 rpm (Biofuge) for one minute, and the supernatant is removed. Two milliliters of sterile distilled water are added to the pellet, and brief sonication is used to resuspend the particles. The suspension is pelleted, one milliliter of absolute ethanol is added to the pellet, and brief sonication is used to resuspend the particles. Rinsing, pelleting, and resuspending of the particles is performed two more times with sterile distilled water, and finally the particles are resuspended in two milliliters of sterile distilled water. The particles are subdivided into 250-ml aliquots and stored frozen.

Preparation of Particle-Plasmid DNA Association:

The stock of tungsten particles are sonicated briefly in a water bath sonicator (Branson Sonifier Model 450, 20% output, constant duty cycle) and 50 ml is transferred to a microfuge tube. All the vectors were cis: that is the selectable marker and the gene of interest were on the same plasmid. These vectors were then transformed either singly or in combination.

Plasmid DNA was added to the particles for a final DNA amount of 0.1 to 10 μg in 10 μL total volume, and briefly sonicated. Preferably,10 μg (1 μg/μL in TE buffer) total DNA is used to mix DNA and particles for bombardment. Fifty microliters (50 ∞L) of sterile aqueous 2.5 M CaCl₂ are added, and the mixture is briefly sonicated and vortexed. Twenty microliters (20 μL) of sterile aqueous 0.1 M spermidine are added and the mixture is briefly sonicated and vortexed. The mixture is incubated at room temperature for 20 minutes with intermittent brief sonication. The particle suspension is centrifuged, and the supernatant is removed. Two hundred fifty microliters (250 μL) of absolute ethanol are added to the pellet, followed by brief sonication. The suspension is pelleted, the supernatant is removed, and 60 ml of absolute ethanol are added. The suspension is sonicated briefly before loading the particle-DNA agglomeration onto macrocarriers.

Preparation of Tissue:

Immature embryos of maize variety High Type II are the targets for particle bombardment-mediated transformation. This genotype is the F₁ of two purebred genetic lines, parents A and B, derived from the cross of two known maize inbreds, A188 and B73. Both parents are selected for high competence of somatic embryogenesis, according to Armstrong et al., Maize Genetics Coop. News 65:92 (1991).

Ears from F₁ plants are selfed or sibbed, and embryos are aseptically dissected from developing caryopses when the scutellum first becomes opaque. This stage occurs about 9-13 days post-pollination, and most generally about 10 days post-pollination, depending on growth conditions. The embryos are about 0.75 to 1.5 millimeters long. Ears are surface sterilized with 20-50% Clorox for 30 minutes, followed by three rinses with sterile distilled water.

Immature embryos are cultured with the scutellum oriented upward, on embryogenic induction medium comprised of N6 basal salts, Eriksson vitamins, 0.5 mg/l thiamine HCl, 30 gm/l sucrose, 2.88 gm/l L-proline, 1 mg/l 2,4-dichlorophenoxyacetic acid, 2 gm/l Gelrite, and 8.5 mg/l AgNO₃. Chu et al., Sci. Sin. 18:659 (1975); Eriksson, Physiol. Plant 18:976 (1965). The medium is sterilized by autoclaving at 121° C. for 15 minutes and dispensed into 100×25 mm Petri dishes. AgNO₃ is filter-sterilized and added to the medium after autoclaving. The tissues are cultured in complete darkness at 28° C. After about 3 to 7 days, most usually about 4 days, the scutellum of the embryo swells to about double its original size and the protuberances at the coleorhizal surface of the scutellum indicate the inception of embryogenic tissue. Up to 100% of the embryos display this response, but most commonly, the embryogenic response frequency is about 80%.

When the embryogenic response is observed, the embryos are transferred to a medium comprised of induction medium modified to contain 120 gm/l sucrose. The embryos are oriented with the coleorhizal pole, the embryogenically responsive tissue, upwards from the culture medium. Ten embryos per Petri dish are located in the center of a Petri dish in an area about 2 cm in diameter. The embryos are maintained on this medium for 3-16 hours, preferably 4 hours, in complete darkness at 28° C. just prior to bombardment with particles associated with plasmid DNAs containing the selectable marker gene/s and structural gene/s of interest.

To effect particle bombardment of embryos, the particle-DNA agglomerates are accelerated using a DuPont PDS-1000 particle acceleration device. The particle-DNA agglomeration is briefly sonicated and 10 ml are deposited on macrocarriers and the ethanol is allowed to evaporate. The macrocarrier is accelerated onto a stainless-steel stopping screen by the rupture of a polymer diaphragm (rupture disk). Rupture is effected by pressurized helium. The velocity of particle-DNA acceleration is determined based on the rupture disk breaking pressure. Rupture disk pressures of 200 to 1800 psi are used, with 650 to 1100 psi being preferred, and about 900 psi being most highly preferred. Multiple disks are used to effect a range of rupture pressures.

The shelf containing the plate with embryos is placed 5.1 cm below the bottom of the macrocarrier platform (shelf #3). To effect particle bombardment of cultured immature embryos, a rupture disk and a macrocarrier with dried particle-DNA agglomerates are installed in the device. The He pressure delivered to the device is adjusted to 200 psi above the rupture disk breaking pressure. A Petri dish with the target embryos is placed into the vacuum chamber and located in the projected path of accelerated particles. A vacuum is created in the chamber, preferably about 28 in Hg. After operation of the device, the vacuum is released and the Petri dish is removed.

Bombarded embryos remain on the osmotically-adjusted medium during bombardment, and 1 to 4 days subsequently. The embryos are transferred to selection medium comprised of N6 basal salts, Eriksson vitamins, 0.5 mg/l thiamine HCl, 30 gm/l sucrose, 1 mg/l 2,4-dichlorophenoxyacetic acid, 2 gm/l Gelrite, 0.85 mg/l Ag NO₃ and 3 mg/l bialaphos (Herbiace, Meiji). Bialaphos is added filter-sterilized. The embryos are subcultured to fresh selection medium at 10- to 14-day intervals. After about 7 weeks, embryogenic tissue, putatively transformed for both selectable marker gene/s and structural gene/s of interest, proliferates from about 7% of the bombarded embryos. Putative transgenic tissue is rescued, and that tissue derived from individual embryos is considered to be an event and is propagated independently on selection medium. Two cycles of clonal propagation are achieved by visual selection for the smallest contiguous fragments of organized embryogenic tissue.

A sample of tissue from each event is processed to recover DNA. The DNA is restricted with a restriction endonuclease and probed with primer sequences designed to amplify DNA sequences overlapping at least a portion of a root-preferred promoter element. Embryogenic tissue with amplifiable sequence is advanced to plant regeneration.

For regeneration of transgenic plants, embryogenic tissue is subcultured to a medium comprising MS salts and vitamins (Murashige & Skoog, Physiol. Plant 15: 473 (1962)), 100 mg/l myo-inositol, 60 gm/l sucrose, 3 gm/l Gelrite, 0.5 mg/l zeatin, 1 mg/l indole-3-acetic acid, 26.4 ng/l cis-trans-abscissic acid, and 3 mg/l bialaphos in 100×25 mm Petri dishes, and is incubated in darkness at 28° C. until the development of well-formed, matured somatic embryos can be seen. This requires about 14 days. Well-formed somatic embryos are opaque and cream-colored, and are comprised of an identifiable scutelium and coleoptile. The embryos are individually subcultured to a germination medium comprising MS salts and vitamins, 100 mg/l myo-inositol, 40 gm/l sucrose and 1.5 gm/l Gelrite in 100×25 mm Petri dishes and incubated under a 16 hour light:8 hour dark photoperiod and 40 meinsteinsm⁻²sec⁻¹ from cool-white fluorescent tubes. After about 7 days, the somatic embryos have germinated and produced a well-defined shoot and root. The individual plants are subcultured to germination medium in 125×25 mm glass tubes to allow further plant development. The plants are maintained under a 16 hour light:8 hour dark photoperiod and 40 meinsteinsm⁻²sec⁻¹ from cool-white fluorescent tubes. After about 7 days, the plants are well-established and are transplanted to horticultural soil, hardened off, and potted into commercial greenhouse soil mixture and grown to sexual maturity in a greenhouse. An elite inbred line is used as a male to pollinate regenerated transgenic plants.

Agrobacterium-mediated Transformation:

As a preferred alternative to particle bombardment, plants are transformed using Agrobacterium-mediated transformation. When Agrobacterium-mediated transformation is used the method of Zhao is employed (PCT patent publication WO98/32326, the contents of which are hereby incorporated by reference). Briefly, immature embryos are isolated from maize and the embryos contacted with a suspension of Agrobacterium (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step) and preferably calli grown on selective medium are cultured on solid medium to regenerate the plants. Regenerated plants are monitored and scored for the activity of the gene of interest.

All publications and patent applications mentioned in the specification are indicative of the level of those skilled in the art to which this invention pertains. All publications and patent applications herein are incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A plant promoter comprising at least one tissue-preferred plant promoter element, said element identified by:
a) providing a first mixture of oligonucleotides each comprising a 5′ flanking sequence, a central random sequence, and a 3′ flanking sequence;
b) contacting said first mixture with a second mixture comprising nuclear proteins from a preferred plant tissue under binding conditions promoting complex formation between said oligonucleotides and said proteins;
c) separating said formed complexes electrophoretically;
d) isolating said separated complexes in ranges of electrophoretic mobility;
e) amplifying oligonucleotides of said isolated complexes by polymerase chain reaction utilizing primers to said flanking sequences;
f) providing said amplified oligonucleotides from step e) as the first mixture for a repetition of step a);
g) performing at least a second cycle of steps b-e with said provided oligonucleotides of step f);
h) assessing for a particular range of electrophoretic mobility and quantity of complex formation in progressive cycles of step g);
i) isolating oligonucleotides of a particular range of electrophoretic mobility wherein said range has increased complex formation in step h);
j) operably linking individual oligonucleotides of step i) to a promoter that drives expression in a plant cell, said promoter operably linked to a coding sequence in an expression cassette;
k) assessing tissue-preferred expression of said coding sequence; and
l) determining sequence of an oligonucleotide having tissue-preferred expression in step k).

2. The promoter of claim 1, wherein said tissue-preferred promoter element is a root-preferred promoter element.

3. The plant promoter of claim 1 comprising at least one synthetic root-preferred plant promoter element that enhances expression of a coding sequence operably linked to said promoter.

4. The plant promoter of claim 1 comprising at least one synthetic root-preferred plant promoter element that suppresses expression of a coding sequence operably linked to said promoter.

5. A plant promoter comprising at least one root-preferred plant promoter element comprising a nucleotide sequence selected from the group consisting of:
a) a nucleotide sequence of SEQ ID NO.1, SEQ ID NO.2, SEQ ID NO.3, SEQ ID NO.4, SEQ ID NO.5, SEQ ID NO.6, SEQ ID NO.7, or SEQ ID NO.8;
b) a nucleotide sequence that hybridizes under stringent conditions to a nucleotide sequence of a); and
c) a nucleotide sequence comprising at least 7 contiguous nucleotides of a sequence of a), wherein said contiguous nucleotides maintain function of the nucleotide sequence of a).

6. A chimeric gene comprising the promoter of claim 5 operably linked to a nucleotide coding sequence of interest.

7. An expression cassette comprising the chimeric gene of claim 6.

8. A transformation vector comprising the expression cassette of claim 7.

9. A transformed plant having stably incorporated into its genome the transformation vector of claim 8.

10. A plant promoter comprising at least one multimeric root-preferred promoter element comprising at least two root-preferred promoter elements further comprising a nucleotide sequence selected from the group consisting of:
a) a nucleotide sequence of SEQ ID NO.1, SEQ ID NO.2, SEQ ID NO.3, SEQ ID NO.4, SEQ ID NO.5, SEQ ID NO.6, SEQ ID NO.7, or SEQ ID NO.8;
b) a nucleotide sequence that hybridizes under stringent conditions to a nucleotide sequence of a); and
c) a nucleotide sequence comprising at least 7 contiguous nucleotides of a sequence of a), wherein said contiguous nucleotides maintain function of the nucleotide sequence of a).

11. A plant promoter comprising at least one root-preferred plant promoter element that enhances expression of a coding sequence operably linked to said promoter, wherein said element comprises a nucleotide sequence selected from the group consisting of:
a) a nucleotide sequence of SEQ ID NO.1, SEQ ID NO.2, SEQ ID NO.3, SEQ ID NO.4, SEQ ID NO.5, SEQ ID NO.6, SEQ ID NO.7, or SEQ ID NO.8;
b) a nucleotide sequence that hybridizes under stringent conditions to a nucleotide sequence of a); and
c) a nucleotide sequence comprising at least 7 contiguous nucleotides of a sequence of a), wherein said contiguous nucleotides maintain function of the nucleotide sequence of a).

12. A plant promoter comprising at least one root-preferred plant promoter element that suppresses expression of a coding sequence operably linked to said promoter, wherein said element comprises a nucleotide sequence selected from the group consisting of:
a) a nucleotide sequence of SEQ ID NO.1, SEQ ID NO.2, SEQ ID NO.3, SEQ ID NO.4, SEQ ID NO.5, SEQ ID NO.6, SEQ ID NO.7, or SEQ ID NO.8;
b) a nucleotide sequence that hybridizes under stringent conditions to a nucleotide sequence of a); and
c) a nucleotide sequence comprising at least 7 contiguous nucleotides of a sequence of a), wherein said contiguous nucleotides maintain function of the nucleotide sequence of a).

13. A transformed plant, or its parts, having stably incorporated into its genome a DNA construct comprising a plant promoter operably linked to a coding sequence, said plant promoter comprising at least one synthetic root-preferred plant promoter element.

14. The plant, or its parts, of claim 13 wherein said element comprises a nucleotide sequence selected from the group consisting of:
a) a nucleotide sequence of SEQ ID NO.1, SEQ ID NO.2, SEQ ID NO.3, SEQ ID NO.4, SEQ ID NO.5, SEQ ID NO.6, SEQ ID NO.7, or SEQ ID NO.8;
b) a nucleotide sequence that hybridizes under stringent conditions to a nucleotide sequence of a); and
c) a nucleotide sequence comprising at least 7 contiguous nucleotides of a sequence of a), wherein said contiguous nucleotides maintain function of the nucleotide sequence of a).

15. The plant, or its parts, of claim 13, wherein said plant is a dicot.

16. The plant, or its parts, of claim 13, wherein said plant is a monocot.

17. The plant, or its parts, of claim 16, wherein said monocot is maize.

18. The plant of claim 13, wherein said plant expresses a DNA coding sequence operably linked to said promoter.

19. A transformed plant cell, said plant cell having stably incorporated into its genome a DNA construct comprising a plant promoter operably linked to a coding sequence, said plant promoter comprising at least one synthetic root-preferred plant promoter element.

20. A method for root-preferred expression of a nucleotide coding sequence in a plant, said method comprising transforming a plant cell with a transformation vector comprising an expression cassette, said expression cassette comprising a plant promoter operably linked to said nucleotide coding sequence, said plant promoter comprising at least one synthetic root-preferred plant promoter element.

21. The method of claim 20 wherein said element comprises a nucleotide sequence selected from the group consisting of:
a) a nucleotide sequence of SEQ ID NO.1, SEQ ID NO.2, SEQ ID NO.3, SEQ ID NO.4, SEQ ID NO.5, SEQ ID NO.6, SEQ ID NO.7, or SEQ ID NO.8;
b) a nucleotide sequence that hybridizes under stringent conditions to a nucleotide sequence of a); and
c) a nucleotide sequence comprising at least 7 contiguous nucleotides of a sequence of a), wherein said contiguous nucleotides maintain function of the nucleotide sequence of a).

22. A method for identifying and isolating tissue-preferred promoter elements, said method comprising the steps of:
a) providing a first mixture of oligonucleotides each comprising a 5′ flanking sequence, a central random sequence, and a 3′flanking sequence;
b) contacting said first mixture with a second mixture comprising nuclear proteins from a preferred plant tissue under binding conditions promoting complex formation between said oligonucleotides and said proteins;
c) separating said formed complexes electrophoretically;
d) isolating said separated complexes in ranges of electrophoretic mobility;
e) amplifying oligonucleotides of said isolated complexes by polymerase chain reaction utilizing primers to said flanking sequences;
f) providing said amplified oligonucleotides from step e) as the first mixture for a repetition of step a);
g) performing at least a second cycle of steps b-e with said provided oligonucleotides of step f);
h) assessing for a particular range of electrophoretic mobility and quantity of complex formation in progressive cycles of step g);
i) isolating by cloning, individual oligonucleotides of a particular range of electrophoretic mobility wherein said range has increased complex formation in step h);
j) simultaneous with step i) or as an individual step, operably linking isolated individual oligonucleotides of step i) to a promoter that drives expression in a plant cell, said promoter operably linked to a coding sequence in an expression cassette;
k) assessing tissue-preferred expression of said coding sequence; and
l) determining sequence of an oligonucleotide having tissue-preferred expression in step k).

23. The method of claim 22 further comprising assessing binding affinity of an individually cloned oligonucleotide of said isolated oligonucleotides of step i) for nuclear proteins from said preferred plant tissue of step b).
